(19) United States
(12) Patent Application Publication
(10) Pub. No.: US 2024/0390099 A1
(43) Pub. Date: Nov. 28, 2024

(54) Surgical Microscope System and Corresponding System, Method and Computer Program for a Surgical Microscope System

(71) Applicant: Leica Instruments (Singapore) Pte. Ltd., Singapore (SG)

(72) Inventors: Jie HUANG, Singapore (SG); Kheng Swee LECK, Singapore (SG)

(21) Appl. No.: 18/693,973

(22) PCT Filed: Sep. 19, 2022
(86) PCT No.: PCT/EP2022/075956

§ 371 Date: Mar. 21, 2024

(30) Foreign Application Priority Data
Sep. 22, 2021 (DE) 102021124579.6

(51) Int. Cl. A61B 90/20; A61B 34/00; A61B 90/00; G06V 10/70; G06V 20/52

(52) U.S. Cl. CPC A61B 90/20; A61B 34/25; G06V 10/70; G06V 20/52; A61B 2034/252; A61B 2034/254; A61B 2090/364; G06V 2201/03

(57) ABSTRACT

Examples relate to a surgical microscope system, and to a corresponding system, method, and computer program for a surgical microscope system. The system comprises one or more processors and one or more storage devices. The system is configured to track a progress of a surgical procedure. The system is configured to select two or more of a plurality of functionalities of the surgical microscope system based on the progress of the surgical procedure. The system is configured to assign the two or more functionalities to two or more input modalities of the surgical microscope system. The system is configured to generate a visual overlay with a visual representation of the two or more functionalities. The two or more functionalities are shown in relation to a visual representation of the two or more input modalities. The system is configured to provide a display signal to a display device of the surgical microscope system, with the display signal comprising the visual overlay.

### TECHNICAL FIELD

Examples relate to a surgical microscope system, and to a corresponding system, method, and computer program for a surgical microscope system.

### BACKGROUND

In general, surgical microscope systems are used in an operation theatre. In most cases, assistants join the surgeon in the operation theater, assisting the main surgeon to carry out the surgical procedure. The assistance ranges from assisting surgical operation, passing medical instruments, to adjusting medical equipment configurations. A typical flow may include the main surgeon telling the chief assistant of the immediate actions needed, for instance, to increase the light intensity, or to hand over the lancet, scissors, etc., followed by the assistant performing the action. The assistants are required to have sufficient experience and knowledge in the surgery procedure and the patient's medical conditions, so that the communication time between surgeon and assistant can be minimized.

In the case where the surgeon has to adjust the equipment by themself, for instance, when the operation theater is not spacious to accommodate extra medical staff other than the surgeon themself, time is taken away from applying treatment to the patient. To help the surgeon in such circumstances, surgeons are allowed to have up to 10 system configurations pre-set. During the surgical procedure, either the surgeon or the assistant can trigger the preset configuration to issue the command through graphical user interface (GUI), or via footswitch, as and when is necessary.

While such pre-set configurations are a valuable help to the surgeon, they require triggering by the surgeon or assistant through the GUI or footswitch. The surgeon or the assistant may be distracted from the main activities at that moment. Moreover, generally, only a limited number (e.g., 10) pre-set configurations may be supported for each user. This may limit the utility that can be provided to the surgeon. For the surgeon/assistant to trigger the appropriate pre-set configurations, they have to be very familiarized with an identifier of each setting, which may require practice and experience with the equipment. If the wrong configuration is triggered, additional time may be required to rectify to the appropriate configuration. Moreover, in case the surgeon wants to have a modified configuration, even just by the slightest amount, extra commands may be required.

There may be a desire for an improved concept for a surgical microscope system, which provides additional assistance to the surgeon during a surgical procedure.

### SUMMARY

This desire is addressed by the subject-matter of the independent claims.

Various embodiments of the present disclosure are based on the finding, that many tasks being performed by surgeons during a surgical procedure lead to changes in settings or to functions being triggered at the surgical microscope system being used, and that many surgical procedures follow a strict sequence of steps. In general, this leads to a sequence of functionalities and/or settings of the surgical microscope system being commonly selected/triggered as the surgical procedure progresses, with the same sequence of functionalities and/or settings being used (by a particular surgeon) in many surgical procedures of the same type. The proposed concept is based on tracking the progress of the surgical procedure, selecting functionalities of the surgical microscope system that are particularly relevant at the current progress of the surgical procedure, and facilitating an access to these functionalities for the surgeon.

An aspect of the present disclosure relates to a system for a surgical microscope system. The system comprises one or more processors and one or more storage devices. The system is configured to track a progress of a surgical procedure. The system is configured to select two or more of a plurality of functionalities of the surgical microscope system based on the progress of the surgical procedure. The system is configured to assign the two or more functionalities to two or more input modalities of the surgical microscope system. The system is configured to generate a visual overlay with a visual representation of the two or more functionalities. The two or more functionalities are shown in relation to a visual representation of the two or more input modalities. The system is configured to provide a display signal to a display device of the surgical microscope system, with the display signal comprising the visual overlay. By tracking the progress of the surgical procedure, a determination can be made on which functionalities are likely to be required at the respective step of the progress. By assigning the two or more functionalities to the two or more input modalities, and providing a corresponding visual representation, the surgeon is made aware of the functionalities being identified, with the functionalities being easily accessible to the surgeon, which may reduce the cognitive load of the surgeon and may decrease interruptions due to the surgeon operating the surgical microscope system during the surgical procedure.

In general, the two or more input modalities may be two or more input modalities of a haptic input device being different from the display device of the surgical microscope system. Display-based input devices are often inaccessible for surgeons due to requirements of keeping a sterile operating environment. For example, the two or more input modalities may be two or more input modalities of a foot pedal or of one or more handles of the surgical microscope system. The haptic input device may be arranged at an optics carrier or a foot pedal of the surgical microscope system, providing easy access to the functionality without having to look at the input modalities. In particular, the two or more input modalities may be implemented by a four-way switch of the foot pedal of the surgical microscope system. The four-way switch may thus facilitate access to the identified two or more functionalities, leaving the remaining buttons/switches of the foot pedal for statically assigned functionality.

In some cases, instead of (or in addition to) a haptic input device, voice recognition may be used to trigger the desired functionality. Accordingly, the two or more input modalities may be two or more keywords of a voice recognition-based control mechanism of the surgical microscope system. A voice recognition-based control mechanism assisted by the respective keywords being used shown on the screen may provide easy access to the functionality without having to avert the gaze from the surgical site.

In the proposed concept, the selected two or more functionalities are based on the progress of the surgical procedure. Accordingly, the system may be configured to update the selection of the two or more functionalities based on the progress of the surgical procedure. This may ensure that the selected two or more functionalities are relevant at the current progress of the surgical procedure.

In some examples, the system is configured to determine a ranking of functionalities with respect to their relevance at a current step of the progress of the surgical procedure, and to select the two or more functionalities based on the ranking. The ranking may be useful in scenarios where different types of criteria (e.g., progress of the surgical procedure and personal preference of the surgeon) are to be reconciled.

For example, the two or more functionalities may be selected based on a deterministic assignment between the progress of the surgical procedure and functionalities of the plurality of functionalities. In other words, for each step of the surgical procedure, two or more functionalities may be deterministically assigned (i.e., assigned in a predetermined manner). This may facilitate an implementation of the feature.

Alternatively, the two or more functionalities may be selected using a machine-learning model being trained to rank the plurality of functionalities based on the progress of the surgical procedure. This may allow for the selection of the two or more functionalities based on the personal preference of the surgeon. Consequently, the machine-learning model being trained to rank the plurality of functionalities may be trained based on a personal preference of a surgeon using the surgical microscope system, such that the two or more functionalities are selected based on a personal preference of the surgeon. This may increase the acceptance and usefulness of the selection of the two or more functionalities, helping the surgeon based on their modus operandi.

There are various approaches to tracking the progress of the surgical procedure. For example, the system may be configured to track the progress of the surgical procedure based on a sequence of commands issued at the surgical microscope system. This is particularly applicable for surgical procedures following a strict surgical plan that mandates the use of a predetermined sequence of commands at the surgical microscope system. For example, the system may be configured to navigate a state machine representing the progress of the surgical procedure based on the sequence of commands issued at the surgical microscope system. The state machine can thus be used to model, and follow, the progress of the surgical procedure.

Alternatively, or additionally, the system may be configured to track the progress of the surgical procedure using a machine-learning model being trained to track the progress of the surgical procedure based on image data of an optical imaging sensor of the surgical microscope system. In other words, object recognition or similar techniques may be used to determine the progress of the surgical procedure, e.g., by recognizing tools being used and/or by recognizing actions being performed.

For example, the surgical procedure may comprise a sequence of steps, with each step comprising one or more tasks being shown in the image data. The machine-learning model may be trained to detect the tasks being shown in the image data. The machine-learning model may be trained to output information on the tasks being shown in the image data. The system may be configured to track the progress of the surgical procedure based on the information on the tasks being output by the machine-learning model. For example, the respective tasks may be distinguishable by the tools being used and/or the actions being performed.

To facilitate interpreting the output of the machine-learning model, each step of the sequence may be assigned an identifier, and the machine-learning model may be trained to output said identifier. Accordingly, the machine-learning model may be trained to output an identifier of the step being shown in the image data. The system may be configured to track the progress of the surgical procedure based on the identifier being provided by the machine-learning model.

For example, the surgical procedure may be an ophthalmic surgical procedure. Ophthalmic surgical procedures, such as cataract surgery, often strictly adhere to a surgical plan.

An aspect of the present disclosure relates to a surgical microscope system comprising the system introduced above.

An aspect of the present disclosure relates to a corresponding method for a surgical microscope system. The method comprises tracking a progress of a surgical procedure. The method comprises selecting two or more of a plurality of functionalities of the surgical microscope system based on the progress of the surgical procedure. The method comprises assigning the two or more functionalities to two or more input modalities of the surgical microscope system. The method comprises generating a visual overlay with a visual representation of the two or more functionalities. The two or more functionalities are shown in relation to a visual representation of the two or more input modalities. The method comprises providing a display signal to a display device of the surgical microscope system, the display signal comprising the visual overlay.

An aspect of the present disclosure relates to a corresponding computer program with a program code for performing the above method when the computer program is executed on a processor.

### SHORT DESCRIPTION OF THE FIGURES

Some examples of apparatuses and/or methods will be described in the following by way of example only, and with reference to the accompanying figures, in which:

FIG. 1a shows a block diagram of an example of a system for a surgical microscope system;

FIG. 1b shows a schematic diagram of an example of a surgical microscope system;

FIG. 2 shows a flow chart of an example of a method for a surgical microscope system;

FIG. 3a shows a schematic drawing of an example of a foot pedal for a surgical microscope system;

FIG. 3b shows a schematic diagram of an example of different input modalities of a foot pedal for a surgical microscope system;

FIG. 4 shows a schematic diagram of a progress of an example of an ophthalmic surgical procedure;

FIGS. 5, 5b and 5c show schematic drawings of an example of the proposed concept; and

FIG. 6 shows a schematic diagram of an example of a system comprising a microscope and a computer system.

### DETAILED DESCRIPTION

Various examples will now be described more fully with reference to the accompanying drawings in which some examples are illustrated. In the figures, the thicknesses of lines, layers and/or regions may be exaggerated for clarity.

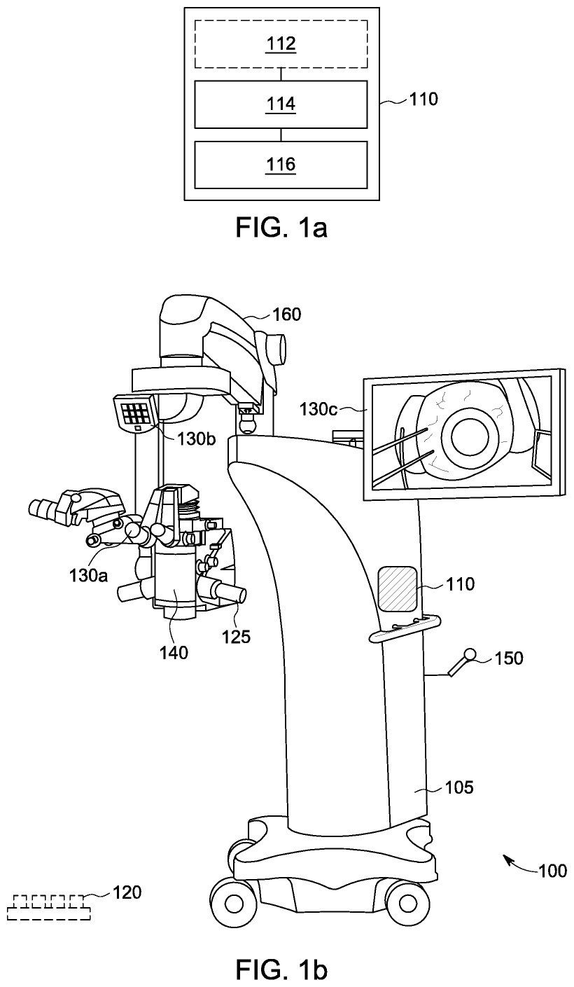

FIG. 1a shows a block diagram of an example of a system 110 for a surgical microscope system 100 (shown in FIG. 1b). The system 110 comprises one or more processors 114 and one or more storage devices 116. Optionally, the system further comprises an interface 112. The one or more processors 114 are coupled to the optional interface 112 and the one or more storage devices 116. In general, the functionality of the system 110 is provided by the one or more processors 114, e.g. in conjunction with the optional interface 112 and/or the one or more storage devices 116.

The system is configured to track a progress of a surgical procedure. The system is configured to select two or more of a plurality of functionalities of the surgical microscope system based on the progress of the surgical procedure. The system is configured to assign the two or more functionalities to two or more input modalities of the surgical microscope system. The system is configured to generate a visual overlay with a visual representation of the two or more functionalities. The two or more functionalities are shown in relation to a visual representation of the two or more input modalities. The system is configured to provide a display signal to a display device 130a; 130b; 130b (shown in FIG. 1b) of the surgical microscope system. The display signal comprises the visual overlay.

Various embodiments of the present disclosure further provide the surgical microscope system 100 comprising the system 110. FIG. 1b shows a schematic diagram of a surgical microscope system 100 for use in ophthalmology comprising the system 110. Accordingly, the surgical procedure may be an ophthalmic surgical procedure. The surgical microscope system 100 further comprises one or more input devices 120; 125, e.g. a foot pedal 120 and/or handles 125, the display device 130, and a microscope 140. The surgical microscope system 100 may comprise one or more additional, optional features, such as a base unit 105 (that comprises the system 110), a microphone 150 (for a voice recognition-based control mechanism), and an arm 160 that the microscope 140 is attached to. The surgical microscope system further comprises one or more display devices 130a-130c. For example, the microscope 140 may comprise ocular eyepieces 130a. Furthermore, the surgical microscope system may comprise one or more auxiliary displays 130b; 130c, which may be arranged at the base unit 105 and/or at a joint between the arm 160 and the microscope 140. The microscope system shown in FIG. 1b is a surgical microscope system, which may be used at a surgical site by a surgeon. In particular, the surgical microscope system shown in FIG. 1b is a surgical microscope system for use in ophthalmology (eye surgery), the same concept may, however, also be used in other types of surgical microscope systems, such as surgical microscope systems for use in neurosurgery. For example, the surgical microscope system may comprise, or be used in combination, with one or more additional systems, such as an optical coherence tomography (OCT) system (not shown).

Embodiments of the present disclosure relate to a system, a method and a computer program that are suitable for a microscope system, such as the surgical microscope system 100 introduced in connection with FIG. 1b. As has been introduced above, a distinction is made between the microscope 140 and the surgical microscope system 100, with the surgical microscope system comprising the microscope 140 and various components that are used in conjunction with the microscope 140, e.g. a lighting system, an auxiliary display etc. In a microscope system, the actual microscope is often also referred to as the “optics carrier”, as it comprises the optical components of the surgical microscope system. In general, a microscope is an optical instrument that is suitable for examining objects that are too small to be examined by the human eye (alone). For example, a microscope may provide an optical magnification of an object. The microscope may thus comprise one or more optical magnification components that are used to magnify a view on the sample. In modern microscopes, the optical magnification is often provided for a camera or an imaging sensor of the microscope.

There are a variety of different types of microscopes. In examples described in connection with FIGS. 1a and/or 1b, the microscope 140 is part of a surgical microscope system 100, e.g. a microscope to be used during a surgical procedure. Although examples are described in connection with a surgical microscope system, they may also be applied, in a more general manner, to any optical device. For example, the microscope system may be a system for performing material testing or integrity testing of materials, e.g. of metals or composite materials.

The starting point of the proposed concept is the tracking of the surgical procedure by the surgical microscope system, in particular by the system 110. There are different ways of achieving this. In a simple example, the progress of the surgical procedure may be tracked manually by the surgeon, e.g., by checking off the progress on a surgical plan, either via a touch-screen or via voice control, advancing from step/stage to step/stage of the surgical plan. However, in many examples, the progress of the surgical procedure may be tracked without manual intervention. In other words, the progress of the surgical procedure may be tracked without requiring manual/human input.

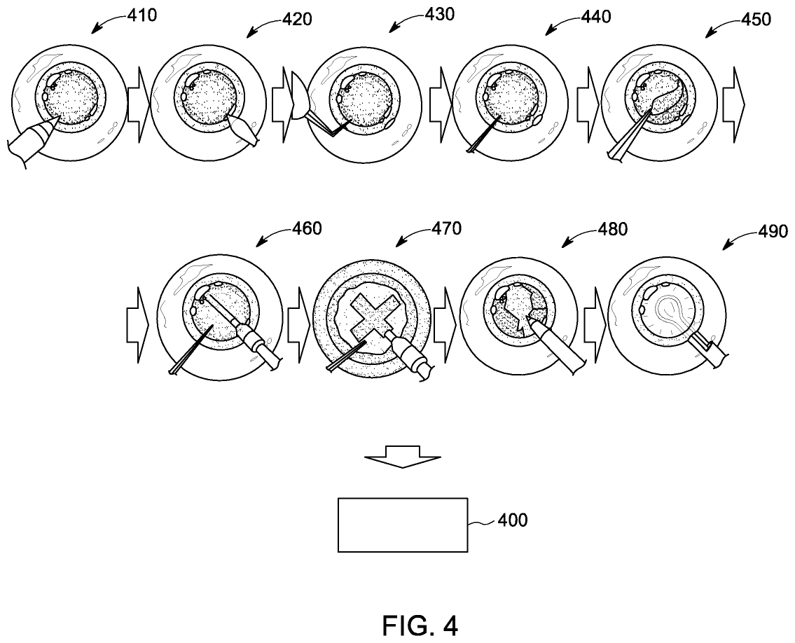

This may be achieved by analyzing signals that are available at the system 110/surgical microscope system 100. In general, two types of signals may be analyzed—signals related to commands being issued at the surgical microscope system (or at a device being coupled to the surgical microscope system), and sensor signals related to one or more sensors of the surgical microscope system. In general, both types of signals may be analyzed in conjunction, as some aspect of the progress can be tracked by identifying the commands, and some aspect of the progress can be tracked by identifying objects or movements in visual sensor data, as will become evident with respect to FIG. 3.

For example, the system may be configured to track the progress of the surgical procedure based on a sequence of commands issued at the surgical microscope system. For example, as outlined in detail in connection with FIG. 3, each step (or some steps) of a sequence of steps of a surgical procedure may be associated with one or more commands being issued at the surgical microscope system, or at an auxiliary device that coupled with the surgical microscope system. For example, at an initial step of the surgical procedure, the surgeon may issue various commands to adjust the surgical microscope system to the patient, e.g., with respect to focus, magnification, illumination, video recording etc. At subsequent steps, different commands may be issued. For example, as shown in FIG. 3, during a “viscoat application” step of a cataract surgical procedure, two pump commands may be issued at a pump being coupled with the surgical microscope system. At a “phacoemulsification” stage, a command may be issued at an ultrasonic tool being coupled with the surgical microscope system. Accordingly, the system may be configured to obtain one or more signals from the surgical microscope system and/or from one or more devices being coupled with the surgical microscope system, to detect one or more commands being issued based on the one or more signals, and to track the progress of the surgical procedure based on a sequence of the one or more commands. For example, the sequence of commands may comprise two or more commands, the two or more commands each being one of a command for controlling an aspect of the surgical microscope system (such as magnification, focusing, illumination, imaging mode), and a command for controlling an aspect of a device being coupled with the surgical microscope system (such as an OCT device, a pump, an ultrasonic tool).

In general, a sequence of commands is being used, as some commands can be issued at various steps of the sequence of steps. Depending on the position of the command within the sequence of commands, the appropriate step of the sequence of steps may be identified. For example, the system may be configured to navigate a state machine representing the progress of the surgical procedure based on the sequence of commands issued at the surgical microscope system. For example, the state machine may comprise a plurality of states and a plurality of transitions between states. The transitions between the states may be taken when a command is issued that is indicative of the surgical procedure having transitioned from one step to another, subsequent step.

Additionally or alternatively, machine-learning may be used to track the progress of the surgical procedure. In other words, the system may be configured to track the progress of the surgical procedure using a machine-learning model being trained to track the progress of the surgical procedure based on image data of an optical imaging sensor 140 of the surgical microscope system. Embodiments may thus be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, meta data and/or image data) may be preprocessed to obtain a feature vector, which is used as input to the machine-learning model.

In the proposed concept, machine-learning may be used to identify tasks that are indicative of a step of the sequence of steps of the surgical procedure. In other words, the machine-learning model may be trained to recognize tasks that are indicative of a step in the image data of the optical imaging sensor of the microscope. For example, each step of the sequence of steps of the surgical procedure may comprise one or more tasks being shown (represented) in the image data. Accordingly, the machine-learning model may be trained to detect the tasks being shown in the image data, and to output information on the tasks being shown in the image data. In turn, the system may be configured to track the progress of the surgical procedure based on the information on the tasks being output by the machine-learning model.

Such a training may be performed using different techniques. Machine-learning models may be trained using training input data. The examples specified with respect to the general introduction of machine-learning use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training.

In the proposed concept, supervised leaning may be used to train the machine-learning model being trained to track the progress of the surgical procedure. For example, still images and/or videos of recordings of the surgical procedure may be used as input data values for the training, and a desired output value, e.g., an indicator of the task being performed, the location of an object indicating a task being performed, the location of a motion (or action) indicating a task being performed etc., may be applied as well. For example, for a still image, an identifier and a location of each object indicating a task being performed may be supplied. For a video, an identifier and a location of each object indicating a task being performed may be supplied together with a timestamp (identifying a point in time or a time interval), and an identifier and a location of each motion (or action) indicating a task being performed may be supplied together with timestamp (identifying a point in time or a time interval) as desired output values. For example, the machine-learning model may be trained to perform image segmentation, feature extraction and object detection or classification on the segmented images. For example, multiple machine-learning models may be used, and possibly trained, together, such as a Dense Trajectory/ST-CNN (Spatial-Temporal-Convolutional Neural Network) for feature extraction and RNN (Recurrent Neural Network) for classification. The machine-learning model may be trained to output an output corresponding to the desired output when supplied with the still images and/or videos of the recordings of the surgical procedure. For example, the machine-learning model may be trained to output an identifier of the object or motion (or action) indicating a task being performed or an output vector comprising binary values for each object or motion (or action) the machine-learning model is being trained to detect.

Apart from supervised learning, semi-supervised learning may be used. In semi-supervised learning, some of the training samples lack a corresponding desired output value. Supervised learning may be based on a supervised learning algorithm (e.g. a classification algorithm, a regression algorithm or a similarity learning algorithm. Classification algorithms may be used when the outputs are restricted to a limited set of values (categorical variables), i.e. the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards). Instead of supervised learning, or in addition to supervised learning, reinforcement learning may be used to train the machine-learning model being trained to track the progress of the surgical procedure, by using the still images/videos of recordings of the surgical procedure as input to the machine-learning model, and by defining a reward function that represents a deviation between the desired output values and the output generated by the machine-learning model.

In the previous examples, the output of the machine-learning model has been described with respect to the task being performed, i.e., the output of the machine-learning model may represent the task being performed (e.g., via the object and/or motion being indicative of the task). In some examples, the training of the machine-learning model may go further, with the output of the machine-learning model representing the step being shown in the image data (with the step being based on the object/motion being detected in the image data). In other words, the machine-learning model may be trained to output an identifier of the step being shown in the image data, with the system being configured to track the progress of the surgical procedure based on the identifier being provided by the machine-learning model. For example, one or more additional layers may be added to the machine-learning model and trained to transform the output on the tasks being performed to the corresponding step of the sequence of steps.

The progress of the surgical procedure is subsequently used to select the two or more functionalities of the surgical microscope system. In particular, the two or more functionalities may be selected based on their suitability at the current step of the surgical procedure. For example, a prediction may be made on what functionalities are likely to be triggered by the surgeon/user of the surgical microscope system, and these functionalities may be selected. For example, the two or more functionalities may be functionalities that are more likely than other functionalities to be triggered by the surgeon/user at the current step of the surgical procedure. These functionalities are then made more easily accessible by the surgeon/user of the surgical microscope system.

The two or more functionalities are selected from the plurality of functionalities of the surgical microscope system. In general, the plurality of functionalities of the surgical microscope system may relate to functionalities of the surgical microscope system and/or settings of the surgical microscope system that can be triggered by the surgeon/user. In other words, any functionality that can be triggered by the surgeon/user, or any setting that can be changed by the user, may be part of the plurality of functionalities. In this regard, the “functionality” may be considered anything that can be controlled/triggered via the surgical microscope system. For example, the plurality of functionalities may comprise one or more functionalities related to controlling an aspect of the surgical microscope system, such as a functionality related to controlling a magnification, a functionality related to controlling a focus, a functionality related to controlling an illumination, a functionality related to controlling a working distance, or a functionality related to controlling an imaging mode (e.g., visual image or OCT image).

In some examples, the plurality of functionalities may comprise functionalities of one or more devices being coupled with the surgical microscope system, such as a pump, an OCT device, an ultrasonic device etc. These devices may be controlled via the surgical microscope system, and/or used in conjunction with the surgical microscope system, and thus also be considered to be functionalities of the surgical microscope system. Therefore, the plurality of functionalities may comprise a functionality related to controlling an auxiliary device, such as an OCT device, a pump, or an ultrasonic device.

In general, the plurality of functionalities may be limited to functionalities that are deemed to be relevant for the (overall) surgical procedure. This may include the basic functionalities of the surgical microscope system and exclude functionalities of auxiliary devices or components not commonly used during the particular surgical procedure.

In some examples, a predetermined (i.e., deterministic) assignment between progress of the surgical procedure and functionality being selected may be used. In other words, the two or more functionalities are selected based on a deterministic assignment between the progress of the surgical procedure and functionalities of the plurality of functionalities. For each step of the sequence of steps of the surgical procedure, two or more functionalities may be defined that are to be assigned to the two or more input modalities.

Alternatively, different, more nuanced approaches may be used, e.g., to determine a contextual ranking of functionalities based on their relevance to the current step of the progress of the surgical procedure. Accordingly, the system may be configured to determine a ranking of functionalities with respect to their relevance at a current step of the progress of the surgical procedure, and to select the two or more functionalities based on the ranking. For example, the two or more highest-ranked functionalities may be selected from the ranking. The ranking may represent the relevance of the respective functionalities at the current step of the progress of the surgical procedure. For example, some basic functionalities, e.g., with respect to focusing and illumination, might always be considered to be relevant, while some functionalities, such as functionalities related to auxiliary devices being coupled to the surgical microscope system, might only be considered relevant at some steps of the sequence of steps. This may lead to the proposed ranking.

For example, machine-learning may be used to determine the ranking. In other words, the two or more functionalities may be selected using a machine-learning model being trained to rank the plurality of functionalities based on the progress of the surgical procedure. When using machine-learning, a non-deterministic selection may be made, which may be based, in part, on personal preferences of the surgeon, or based on practice in the hospital hosting the surgical microscope system. For example, the machine-learning model being trained to rank the plurality of functionalities may be trained based on a personal preference of a surgeon using the surgical microscope system, such that the two or more functionalities are selected based on a personal preference of the surgeon. Additionally or alternatively, the machine-learning model being trained to rank the plurality of functionalities may be trained based on a practice being used in a hospital hosting the surgical microscope system, such that the two or more functionalities are selected based on the practice being used in the hospital. In the first case, the selection can be specifically tailored to the surgeon, which may require separate training of the machine-learning model for each surgeon (or the training of different machine-learning models for each surgeon). In the latter case, a general practice within the hospital may be applied, which may be followed by the surgeons of the hospital. Again, supervised learning may be used to train the machine-learning model, using information on the progress of the surgical procedure as input data values, and using a desired ranking of functionalities, or values representing a suitability of the functionalities at the current step of the progress of the surgical procedure as desired output values. The desired output values may be collected during use of the surgical microscope system, and the training of the machine-learning model may be adapted when new desired output values are collected.

The selection of the two or more functionalities may be kept up-to-date as the surgical procedure progresses. Accordingly, the system may be configured to update the selection of the two or more functionalities based on the progress of the surgical procedure. Consequently, the system may also be configured to update the assignment and the visual overlay based on the updated selection.

The selected two or more functionalities are then assigned to two or more input modalities of the surgical microscope system. In this context, a distinction is made between “input devices” and “input modalities”. In general, an “input device” may relate to the device itself, such as a foot pedal, or a handle, and the “input modality” may relate to the means provided by the input device to trigger a functionality. For example, an input device may comprise a plurality of input modalities, with the plurality of input modalities comprising at least one of one or more buttons, one or more switches, one or more rotary controls, and one or more control sticks. For example, the input device “foot pedal” 120 may comprise a plurality of input modalities, e.g. buttons that can be pressed, pads that can be tilted in one or another direction, or control sticks that can be tilted in one of multiple directions. For example, in FIG. 3b, a foot pedal is shown (i.e. the input device), with six buttons/switches 301, 302, 307, 308, 309, 310, two two-way switches 303/304 and 305/306, and a four-way switch 311-314. For example, if the input device is an eye-tracking system, the different directions of the gaze of the user may correspond to the input modalities. If the input device is a mouth switch, the input modalities may be an “actuation modality” (e.g. a switch to be actuated via the chin), and a four-way joystick to be actuated via the mouth. If the input device, is a voice recognition-based control mechanism 150, the input modalities may be different keywords being used to trigger the functionalities.

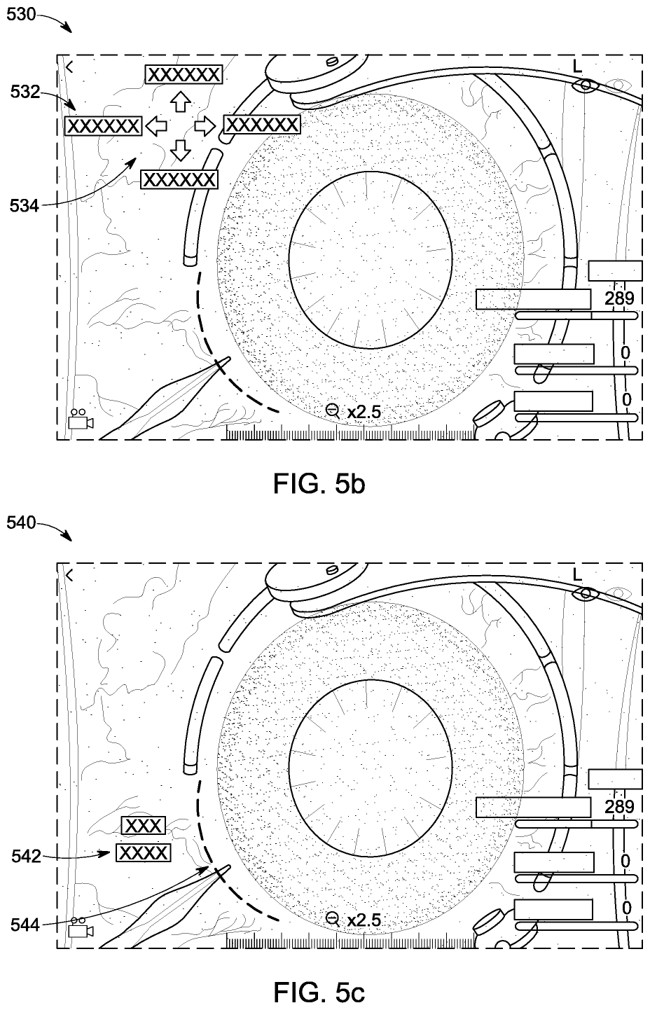

In various examples, the two or more input modalities may be two or more input modalities of a haptic input device 120; 125, such as the foot pedal 120, a handle 125, or a mouth switch (not shown). For example, the two or more input modalities may be two or more input modalities of a foot pedal 120, of one or more handles 125, or of a mouth switch of the surgical microscope system. As shown in FIGS. 3b, 5a and 5b, the two or more input modalities may be implemented by a four-way switch of the foot pedal of the surgical microscope system, for example. In this context, the term “haptic input device” indicates, that a switch, button, pedal, stick etc. is haptically actuated. For example, the term “haptic input device” may exclude similar input devices, such as touch screen. For example, the haptic input device may be different from the display device of the surgical microscope system being used to display the display signal.

Alternatively, a non-haptic input device may be used. For example, voice recognition may be used to trigger the two or more functionalities. Accordingly, the two or more input modalities may be two or more keywords of a voice recognition-based control mechanism 150 of the surgical microscope system. For the example, the voice recognition-based control mechanism 150 may be implemented by the system 110. In other words, the system may be configured to detect the two or more keywords in an audio signal captured via a microphone of the surgical microscope system.

Based on the selected functionalities and based on the input modalities they are assigned to; a visual overlay is generated. In general, any kind of visual representation of the two or more functionalities may be part of the visual overlay. For example, the visual representation may comprise a pictogram representation of the two or more functionalities, or the visual representation may comprise a textual representation of the two or more functionalities. In other words, for each of the two or more functionalities accessible via an input modality of the input modality, the visual representation may comprise a textual or pictogram representation, e.g. as shown in FIG. 5b. In addition, the visual overlay comprises a representation of the input modalities the two or more functionalities are assigned to. For example, a pictogram or rendering representing the input modalities and/or the input device comprising the input modalities may be included in the visual overlay (as further shown in FIG. 5b). The representation of the two or more functionalities of may included such, that the textual or pictogram representation of the respective functionality is arranged adjacent to the respective input modality being used to trigger the functionality. For example, as shown in FIG. 5b, when the two or more functionalities are four functionalities that are assigned to the four directions of a four-way switch, the input modalities may be represented by a pictogram 534 representing the four-way switch, and the assigned functionalities may be represented by a textual representation 532 (indicated by “XXXXXX” in FIG. 5b) that is arranged adjacent to the different directions of the four-way switch. For example, if the two or more input modalities include keywords or key phrases of a voice recognition-based control mechanism, the voice recognition-based control mechanism may be represented by a pictogram (e.g., such as the pictogram 560 shown in FIG. 5a), with the keywords or key phrases being shown alongside the pictogram.

In some examples, instead of the display signal with the visual overlay, or in addition to the display signal with the visual overlay, the two or more functionalities and the two or more associated input modalities (e.g., keywords) may be provided (e.g., announced) via a voice-based user interface. The surgeon/user may trigger the functionalities by triggering the respective input modality at the haptic input device, or by uttering the respective keyword/key phrase.

In some examples, in addition to the two or more functionalities, the proposed system may be used to propose one or more surgical steps based on the progress of the surgical procedure. Similar to the above-referenced selection of the two or more functionalities, the one or more surgical steps may be selected using a deterministic assignment or using a machine-learning model, with the one or more surgical steps being suitable at a current step of the progress of the surgical procedure. For example, the one or more surgical steps may include one or more surgical tasks to be performed by the surgeon and/or one or more proposals for locations for performing the one or more surgical tasks (as shown in FIG. 5c).

The system is configured to provide the display signal to a display device 130 of the surgical microscope system (e.g. via the interface 112), the display signal comprising the visual overlay. The display device may be configured to show the visual overlay based on the display signal, e.g. to inject the visual overlay over the view on the sample based on the display signal. For example, the display signal may comprise a video stream or control instructions that comprise the visual overlay, e.g. such that the visual overlay is shown by the respective display device. For example, the display device may be one of an ocular display 130a of the microscope and an auxiliary display 130b; 130c of the surgical microscope system. In modern surgical microscope systems, the view on the sample is often provided via a display, such as an ocular display, an auxiliary display, or a headset display, e.g. using a video stream that is generated based on image sensor data of an optical imaging sensor of the respective microscope. In this case, the visual overlay may be merely overlaid over the video stream. For example, the system may be configured to obtain image sensor data of an optical imaging sensor of the microscope, to generate the video stream based on the image sensor data and to generate the display signal by overlaying the visual overlay over the video stream.

Alternatively, the visual overlay may be overlaid over an optical view of the sample. For example, the ocular eyepieces of the microscope may be configured to provide an optical view on the sample, and the display device may be configured to inject the overlay into the optical view on the sample, e.g. using a one-way mirror or a semi-transparent display that is arranged within an optical path of the microscope. For example, the microscope may be an optical microscope with at least one optical path. One-way mirror(s) may be arranged within the optical path(s), and the visual overlay may be projection onto the one-way mirror(s) and thus overlaid over the view on the sample. In this case, the display device may be a projection device configured to project the visual overlay towards the mirror(s), e.g. such that the visual overlay is reflected towards an eyepiece of the microscope. Alternatively, a display or displays may be used to provide the overlay within the optical path(s) of the microscope. For example, the display device may comprise at least one display being arranged within the optical path(s). For example, the display(s) may be one of a projection-based display and a screen-based display, such as a Liquid Crystal Display (LCD)—or an Organic Light Emitting Diode (OLED)-based display. For example, the display(s) may be arranged within the eyepiece of the optical stereoscopic microscope, e.g. one display in each of the oculars. For example, two displays may be used to turn the oculars of the optical microscope into augmented reality oculars, i.e. an augmented reality eyepiece. Alternatively, other technologies may be used to implement the augmented reality eyepiece/oculars.

For example, the optical imaging sensor of the microscope may comprise or be an APS (Active Pixel Sensor)—or a CCD (Charge-Coupled-Device)-based imaging sensor. For example, in APS-based imaging sensors, light is recorded at each pixel using a photodetector and an active amplifier of the pixel. APS-based imaging sensors are often based on CMOS (Complementary Metal-Oxide-Semiconductor) or S-CMOS (Scientific CMOS) technology. In CCD-based imaging sensors, incoming photons are converted into electron charges at a semiconductor-oxide interface, which are subsequently moved between capacitive bins in the imaging sensor modules by a control circuitry of the sensor imaging module to perform the imaging. The image data may be obtained by receiving the image data from the optical imaging sensor (e.g. via the interface 112 and/or the system 110), by reading the image data out from a memory of the imaging sensor (e.g. via the interface 112), or by reading the image data from a storage device 116 of the system 110, e.g. after the image data has been written to the storage device 116 by the optical imaging sensor or by another system or processor.

The interface 112 may correspond to one or more inputs and/or outputs for receiving and/or transmitting information, which may be in digital (bit) values according to a specified code, within a module, between modules or between modules of different entities. For example, the interface 112 may comprise interface circuitry configured to receive and/or transmit information. In embodiments the one or more processors 114 may be implemented using one or more processing units, one or more processing devices, any means for processing, such as a processor, a computer or a programmable hardware component being operable with accordingly adapted software. In other words, the described function of the one or more processors 114 may as well be implemented in software, which is then executed on one or more programmable hardware components. Such hardware components may comprise a general-purpose processor, a Digital Signal Processor (DSP), a micro-controller, etc. In at least some embodiments, the one or more storage devices 116 may comprise at least one element of the group of a computer readable storage medium, such as a magnetic or optical storage medium, e.g. a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g. of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input. For example, the machine-learning model being trained to track the progress of the surgical procedure may be a Convolutional Neural Network (CNN), such as a dense trajectory CNN, a spatial-temporal CNN, or a Recurrent Neural Network (RNN). CNNs are particularly suitable for the analysis of image data, with spatial-temporal CNNs providing improved support for the analysis of sequences of image data. RNNs are particularly suitable for the analysis of sequences, such as sequences of tasks.

Alternatively, the machine-learning model may be a support vector machine, a random forest model or a gradient boosting model. Support vector machines (i.e. support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

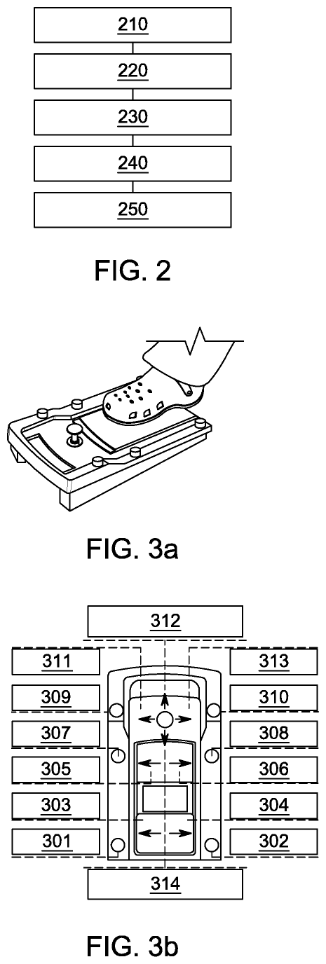

More details and aspects of the system and surgical microscope system are mentioned in connection with the proposed concept or one or more examples described above or below (e.g. FIG. 2 to 6). The system and/or surgical microscope system may comprise one or more additional optional features corresponding to one or more aspects of the proposed concept or one or more examples described above or below.

FIG. 2 shows a flow chart of an example of a corresponding method for a surgical microscope system. The method comprises tracking 210 a progress of a surgical procedure. The method comprises selecting 220 two or more of a plurality of functionalities of the surgical microscope system based on the progress of the surgical procedure. The method further comprises assigning 230 the two or more functionalities to two or more input modalities of the surgical microscope system. The method comprises generating 240 a visual overlay with a visual representation of the two or more functionalities, the two or more functionalities being shown in relation to a visual representation of the two or more input modalities. The method comprises providing 250 a display signal to a display device of the surgical microscope system, the display signal comprising the visual overlay.

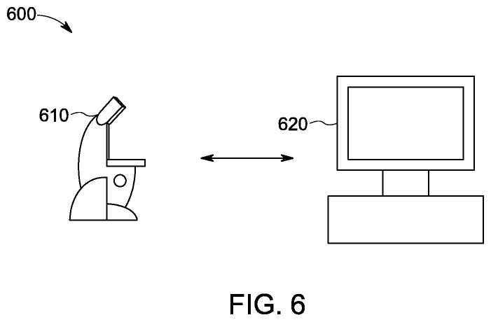

Features described in connection with the system 110 and the surgical microscope system 100 of FIGS. 1a and/or 1b may be likewise applied to the method of FIG. 2. For example, the method may be a computer-implemented method. The method may be performed by the surgical microscope system, e.g., by a computer system of the surgical microscope system, such as the system 110 of FIGS. 1a and/or 1b or by the computer system 620 introduced in connection with FIG. 6

More details and aspects of the method are mentioned in connection with the proposed concept or one or more examples described above or below (e.g. FIG. 1a to 1b, 3a to 6). The method may comprise one or more additional optional features corresponding to one or more aspects of the proposed concept or one or more examples described above or below.

Various examples of the present disclosure relate to an AI (artificial intelligence-based, also machine learning-based) surgical assistant with command recommendation and voice control, with the use of artificial intelligence and voice control being optional.

The proposed surgical assistant is based on tracking the progress of the respective surgical procedure. The surgical assistant may provide real time surgical activity segmentation (to segment the surgical procedure into steps). From there, the surgical assistant may suggest the surgeon with the commands that are most likely to be required by the surgery process at the current timestep, e.g., ranked by probabilities. The recommendation can be prompted through a GUI or through machine speech (i.e., a tech-to-speech system).

Additionally, the suggestions provided may be produced according to the surgical activity that happened immediately before the moment, which can be obtained by neural networks trained on various medical surgical videos. This feature may reduce the requirement on the knowledge level and experience of the chief assistant.

For example, medical video clips and system data output logs may be used to train one or more neural networks (e.g., using Dense Trajectory/ST-CNN (Spatial-Temporal-Convolutional Neural Network) for feature extraction and RNN (Recurrent Neural Network) for classification or using a temporal convolutional network or reinforcement learning) to segment the surgical activities (i.e., to determine the steps of the surgical procedure). Another family of neural networks may be trained to map the segmented surgical activities (i.e., the steps of the surgical procedure) to the system commands (i.e., the two or more functionalities) that might be issued at certain timesteps. The produced recommended commands may be ranked and suggested to the user either through the GUI (e.g., through the visual overlay) or through audio.

In some examples, the triggering of the command may be done through speech. Either the surgeon or the assistant can speak out the command, be it one of the suggestions provided by the AI surgical assistant or other commands, without selecting the configuration setting on the GUI. Thus the feature may save surgeons the possible adverse outcome of being distracted and then re-focusing on the task on hand. More importantly, more time can be saved for the actual surgery. Speech recognition can be done either using cloud-based voice recognition or using one or more pre-trained models that are fine-tuned on medical context, to map a (spoken) user command into a corresponding system command (to trigger a functionality).

As a typical workflow in the proposed concept, the system may prompt the surgeon on possible re-configuration of the system based on the current medical activities. Surgeons can then issue commands through a GUI or voice to trigger the specific adjustment.

In summary, the proposed concept may reduce or eliminate the necessities of having to go over to the GUI for change of system settings, which may lower the knowledge and experience requirement for the assistant, and most importantly save time in the actual surgery.

More details and aspects of the surgical assistant are mentioned in connection with the proposed concept or one or more examples described above or below (e.g. FIG. 1a to 2, 4 to 6). The surgical assistant may comprise one or more additional optional features corresponding to one or more aspects of the proposed concept or one or more examples described above or below.

FIG. 3a shows a schematic drawing of an example of a foot pedal for a surgical microscope system. The foot pedal comprises a plurality of input modalities, such as switches (i.e., buttons), two-way switches and a four-way switch. The input modalities are triggered by the foot of the surgeon.

FIG. 3b shows a schematic diagram of an example of different input modalities of a foot pedal for a surgical microscope system. In FIG. 3a, the foot pedal comprises a bottom left switch 301, a bottom right switch 302, a lower two-way switch (left side 303 and right side 304), an upper two-way switch (left side 305 and right side 306), middle switches left 307 and right 308, top switches left 309 and right 310, and a four-way switch with directions left 311, top 312, right 313, and bottom 314.

In an example of an ophthalmic surgical microscope system, the foot switch (foot pedal—the terms foot switch and foot pedal are used interchangeably in this disclosure) can have multiple modes—anterior mode, anterior OCT mode, VR mode, and VR OCT mode.

For example, in the anterior mode, the bottom left switch 301 may be set to all lights on/off, the bottom right switch 302 may be set to OCT mode on/off, the lower two-way switch 303; 304 may be set to magnification − and + (left and right, respectively), the upper two-way switch 305; 306 may be set to focus − and + (left and right, respectively), the middle left switch 307 and the top left switch 309 may be set to main light − and +, respectively, the middle right switch 308 and the top right switch 310 may be set to red reflex + and −, respectively, and the four-way switch 311-314 may be set to move X− and X+ (left and right) and move Y− and Y+ (bottom and top). In other modes, the configuration may be changed. For example, in the two OCT modes, the switches may be set to control the OCT instead of the microscope.

This multitude of functionalities being assigned to the foot pedal may be considered to not be user friendly, especially for new users. In effect, a surgeon may have difficulty to remember the assignment. Therefore, the user/surgeon may print out the footswitch assignment and past the printed paper onto the surgical microscope system.

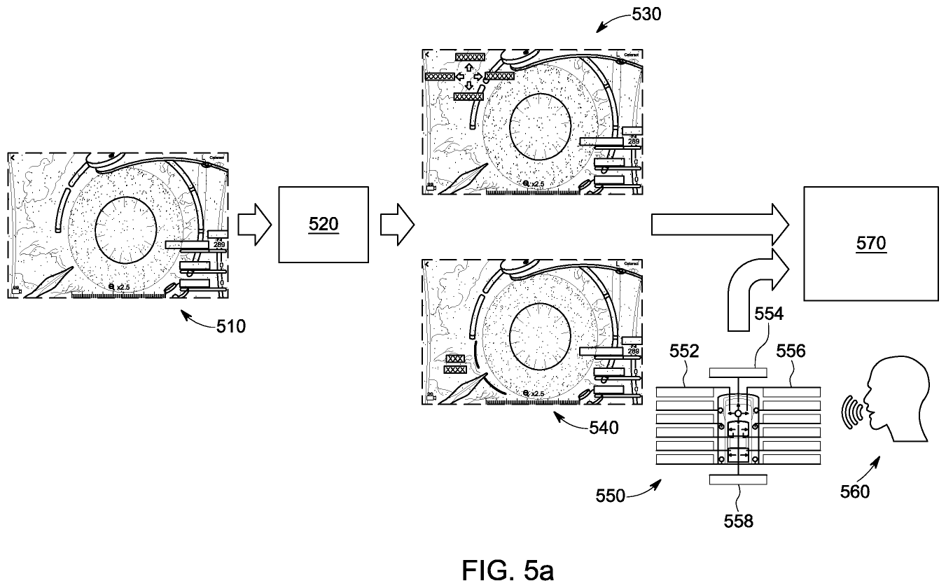

FIG. 4 shows a schematic diagram of a progress of an example of an ophthalmic surgical procedure. The example shown in FIG. 4 shows a cataract surgery, comprising steps 410-490. The different steps can be distinguished based on commands issued by the user/surgeon at the surgical microscope system or at an auxiliary device, objects detected in image data of the surgical procedure, and/or motion detected in the image data.

First a port incision 410 is performed. The port incision can be detected based on one or more of the following: light on (command), focusing (series of commands), magnification adjustment (series of commands), video recording start (command), and paracentesis blade (detected object). The port incision 410 is followed by a second incision 420. The second incision 420 can be detected based on the detection of a keratome blade (detected object). The second incision 420 is followed by viscoat application 430. The viscoat application 430 can be detected based on one or more of viscoat injection (command issued at pump) and anesthetic injection (command issued at pump). The viscoat application 430 is followed by continuous curvilinear capsulorhesis 440, which may be detected based on one or more of cystotome blade (detected object) and anesthetic injection (command issued at pump). The continuous curvilinear capsulorhesis 440 is followed by flap creation 450, which can be detected based on one or more of cystotome blade (detected object), and peel away flap (detected motion). The flap creation 450 is followed by phacoemulsification 460, which may be detected using one or more of phacoemulsifier (detected object) and break down nucleus (ultrasonic command and detected motion). The phacoemulsification 460 is followed by suction 470, which may be detected using one or more of phacoemulsifier (detected object) and suction (suction command and detected motion). The suction 470 is followed by irrigation/aspiration 480, which may be detected based on one or more of irrigation hand piece (detected object), aspiration hand piece (detected object), cleaning process (detected motion), and red-reflex illumination (illumination command). The irrigation/aspiration 480 is followed by intraocular lens insertion, which may be detected based on one or more of lens injector (detected object) and adjustment (detected motion). The actions, objects and motions may be used to train a neural network 400.

FIGS. 5, 5b and 5c show schematic drawings of an example of the proposed concept. In FIG. 5a, the overall flow of the example is shown. It starts at the current stage/step 510 of the surgical procedure (the port incision stage/step in FIG. 5a). Then, a mechanism, such as a trained neural network 520 may be used to predict possible steps (i.e., possible functionalities of the surgical microscope system and/or possible surgical steps). The proposed functions may be shown 530 overlaid over the visual image of the surgical site. In FIG. 5b, the view 530 is shown in more detail, with the proposed functions 532 (a textual description of which is indicated by “XXXXXX”) being shown next to a representation 534 of the input modalities (a four-way foot switch in this case). The proposed surgical steps 540 are shown in view 540, which is shown in more detail in FIG. 5c. FIG. 5c shows the proposed surgical steps 532 (a textual description is indicated by “XXX”), along a proposed incision position 544. The user chooses the desired functionality via input devices/modalities 550; 560, with input device 550 being a foot pedal/footswitch and input device 560 a voice-recognition-based input modality. In FIG. 5a, the four-way switch 552-558 is used for proposed functions 1-4, as indicated in view 530. Upon selection by the user/surgeon, the desired function is triggered 570.

More details and aspects of the surgical microscope system, the system or method for the surgical microscope system, and the corresponding surgical assistant are mentioned in connection with the proposed concept or one or more examples described above or below (e.g. FIG. 1a to 2, 6). The surgical microscope system, the system or method for the surgical microscope system, and the corresponding surgical assistant may comprise one or more additional optional features corresponding to one or more aspects of the proposed concept or one or more examples described above or below.

Some embodiments relate to a microscope comprising a system as described in connection with one or more of the FIGS. 1 to 5c. Alternatively, a microscope may be part of or connected to a system as described in connection with one or more of the FIGS. 1 to 5c. FIG. 6 shows a schematic illustration of a system 600 configured to perform a method described herein. The system 600 comprises a microscope 610 and a computer system 620. The microscope 610 is configured to take images and is connected to the computer system 620. The computer system 620 is configured to execute at least a part of a method described herein. The computer system 620 may be configured to execute a machine learning algorithm. The computer system 620 and microscope 610 may be separate entities but can also be integrated together in one common housing. The computer system 620 may be part of a central processing system of the microscope 610 and/or the computer system 620 may be part of a subcomponent of the microscope 610, such as a sensor, an actor, a camera or an illumination unit, etc. of the microscope 610.

The computer system 620 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 620 may comprise any circuit or combination of circuits. In one embodiment, the computer system 620 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 620 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 620 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 620 may also include a display device, one or more speakers, and a keyboard and/or controller, which can include a mouse, trackball, touch screen, voice-recognition device, or any other device that permits a system user to input information into and receive information from the computer system 620.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a nontransitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

### LIST OF REFERENCE SIGNS

100 Surgical microscope system
110 System
112 One or more interfaces
114 One or more processors
116 One or more storage devices
120 Foot pedal
125 Handles
130a,b,c Display devices
140 Microscope
150 Microphone, voice recognition-based control mechanism
210 Tracking a progress of a surgical procedure
220 Selecting two or more functionalities
230 Assigning the two or more functionalities to two or more input modalities
240 Generating a visual overlay
250 Providing a display signal
301-314 Switches of a foot pedal
400 Trained neural network
410-490 Steps of a cataract surgical procedure
510 Current progress of surgical procedure
520 Trained neural network
530 Visual overlay showing proposed functions
532 Proposed functions
534 Representation of input modalities
540 Visual overlay showing proposed surgical steps
542 Proposed surgical steps
544 Proposed incision position
550 Foot pedal
552-552 Input modalities of foot pedal assigned with proposed functionality
560 Voice control
570 Trigger desired function
600 System
610 Microscope
620 Computer system

1. A system for a surgical microscope system, the system comprising one or more processors and one or more storage devices, wherein the system is configured to:
track a progress of a surgical procedure;
select two or more of a plurality of functionalities of the surgical microscope system based on the progress of the surgical procedure;
assign the two or more functionalities to two or more input modalities of the surgical microscope system;
generate a visual overlay with a visual representation of the two or more functionalities, the two or more functionalities being shown in relation to a visual representation of the two or more input modalities; and
provide a display signal to a display device of the surgical microscope system, the display signal comprising the visual overlay.

2. The system according to claim 1, wherein the two or more input modalities are two or more input modalities of a haptic input device being different from the display device of the surgical microscope system.

3. The system according to claim 1, wherein the two or more input modalities are two or more input modalities of a foot pedal or of one or more handles of the surgical microscope system.

4. The system according to claim 3, wherein the two or more input modalities are implemented by a four-way switch of the foot pedal of the surgical microscope system.

5. The system according to claim 1, wherein the two or more input modalities are two or more keywords of a voice recognition-based control mechanism of the surgical microscope system.

6. The system according to claim 1, wherein the system is configured to update the selection of the two or more functionalities based on the progress of the surgical procedure.

7. The system according to claim 1, wherein the system is configured to determine a ranking of functionalities with respect to their relevance at a current step of the progress of the surgical procedure, and to select the two or more functionalities based on the ranking.

8. The system according to claim 1, wherein the two or more functionalities are selected based on a deterministic assignment between the progress of the surgical procedure and functionalities of the plurality of functionalities.

9. The system according to claim 1, wherein the two or more functionalities are selected using a machine-learning model being trained to rank the plurality of functionalities based on the progress of the surgical procedure.

10. The system according to claim 9, wherein the machine-learning model being trained to rank the plurality of functionalities is trained based on a personal preference of a surgeon using the surgical microscope system, such that the two or more functionalities are selected based on a personal preference of the surgeon.

11. The system according to claim 1, wherein the system is configured to track the progress of the surgical procedure based on a sequence of commands issued at the surgical microscope system.

12. The system according to claim 1, wherein the system is configured to track the progress of the surgical procedure using a machine-learning model being trained to track the progress of the surgical procedure based on image data of an optical imaging sensor of the surgical microscope system.

13. The system according to claim 12, wherein the surgical procedure comprises a sequence of steps, with each step of the sequence of steps comprising one or more tasks being shown in the image data, wherein the machine-learning model is trained to detect the tasks being shown in the image data, and to output information on the tasks being shown in the image data, wherein the system is configured to track the progress of the surgical procedure based on the information on the tasks being output by the machine-learning model.

14. A method for a surgical microscope system, the method comprising:
tracking a progress of a surgical procedure;
selecting two or more of a plurality of functionalities of the surgical microscope system based on the progress of the surgical procedure;
assigning the two or more functionalities to two or more input modalities of the surgical microscope system;
generating a visual overlay with a visual representation of the two or more functionalities, the two or more functionalities being shown in relation to a visual representation of the two or more input modalities; and
providing a display signal to a display device of the surgical microscope system, the display signal comprising the visual overlay.

15. A computer program with a program code for performing the method according to claim 14 when the computer program is executed on a processor.
